(19) United States
(12) Patent Application Publication
(10) Pub. No.: US 2009/0191273 A1
(43) Pub. Date: Jul. 30, 2009

(54) Metal Oxide Dispersion

(75) Inventors: Lorna M. Kessell, North Yorkshire (GB); Benjamin J. Naden, Cleveland (GB)

Correspondence Address: JONES DAY, 222 EAST 41ST ST, NEW YORK, NY, 10017, US

(73) Assignee: Croda International PLC, Goole, North Humberside (GB)

(21) Appl. No.: 11/992,400

(22) PCT Filed: Sep. 20, 2006
(86) PCT No.: PCT/GB2006/003471

§ 371 Date: Mar. 11, 2009

(30) Foreign Application Priority Data
Sep. 23, 2005 (GB) 0519444.4

(51) Int. Cl. A61K 9/14; A61K 8/19; A61Q 17/04

(52) U.S. Cl. 424/489

(57) ABSTRACT

A dispersion contains particles of metal oxide having a median volume particle diameter in the range from 24 to 42 nm which are dispersed in a medium which includes a mixture of at least one polar material having an interfacial tension of less than 30 mNm⁻¹ and at least one siloxane fluid. The dispersion can be used in a sunscreen product that exhibits effective UV protection, transparency, and improved skin feel.

### FIELD OF INVENTION

The present invention relates to a dispersion of metal oxide particles, and in particular to the use thereof in a sunscreen product.

### BACKGROUND

Metal oxides such as titanium dioxide, zinc oxide and iron oxide have been employed as attenuators of ultraviolet light in sunscreens. Due to the increased awareness of the link between ultraviolet light and skin cancer, there has been an increasing requirement for ultraviolet light protection in everyday skincare and cosmetics products. There is a need for a metal oxide in a form which when incorporated into sunscreen products exhibits effective UV absorption properties, be transparent in use, and have a pleasant skin feel. It is an extremely challenging problem to provide a product with all of these properties. There is a constant need in the market place to improve these properties, particularly transparency and skin feel.

The use of siloxane fluids or silicone based oils in cosmetics has become popular because they can produce an improved skin feel. Hence, metal oxide dispersions in siloxane based dispersing media are desirable. Such dispersions have been difficult to produce, particularly at high metal oxide concentrations, and especially at low viscosity. In addition, polysiloxane dispersing agents normally have to be used in siloxane dispersing media, but high concentrations of polysiloxane dispersing agents usually have to be employed, and even then it can be very difficult to obtain a dispersion having a high metal oxide concentration and/or low viscosity.

### SUMMARY OF THE INVENTION

We have now surprisingly discovered an improved metal oxide dispersion, which overcomes or significantly reduces at least one of the aforementioned problems.

Accordingly, the present invention provides a dispersion comprising particles of metal oxide having a median volume particle diameter in the range from 24 to 42 nm, dispersed in a medium which comprises a mixture of (i) at least one polar material having an interfacial tension of less than 30 mNm⁻¹, and (ii) at least siloxane fluid.

The present invention also provides a dispersion comprising particles of metal oxide having a median volume particle diameter in the range from 24 to 42 nm, dispersed in a medium which comprises a mixture of (i) at least one polar material selected from the group consisting of C12-15 alkyl benzoate, caprylic/capric triglyceride, cetearyl isononanoate, ethylhexyl isostearate, ethylhexyl palmitate, isononyl isononanoate, isopropyl isostearate, isopropyl myristate, isostearyl isostearate, isostearyl neopentanoate, octyldodecanol, pentaerythrityl tetraisostearate, PPG-15 stearyl ether, triethylhexyl triglyceride, dicaprylyl carbonate, ethylhexyl stearate, hellanthus annus (sunflower) seed oil, isopropyl palmitate, octyldodecyl neopentanoate, propylene glycol isostearate, cetyl ethylhexanoate, cetearyl octanoate, hexyl laurate, isodecyl neopentanoate, isopropyl palmitate, decyl oleate, and isostearyl alcohol; and (ii) at least one siloxane fluid selected from the group consisting of, dimethylpolysiloxane, dimethyl silicone, highly polymerized methyl polysiloxane, and methyl polysiloxane, cyclic oligomeric dialkylsiloxanes, dimethylsiloxane linear oligomers or polymers, and phenyltris(trimethylsiloxy)silane.

The present invention further provides a sunscreen product formed from a dispersion comprising particles of metal oxide having a median volume particle diameter in the range from 24 to 42 nm, dispersed in a medium which comprises a mixture of (i) at least one polar material having an interfacial tension of less than 30 mNm⁻¹, and (ii) at least one siloxane fluid.

The present invention further provides a sunscreen product comprising particles of metal oxide having a median volume particle diameter in the range from 24 to 42 nm, and (i) at least one polar material selected from the group consisting of isopropyl isostearate and propylene glycol isostearate, and (ii) at least one siloxane fluid selected from the group consisting of cyclomethicone and dimethicone.

The invention yet further provides the use of a dispersion comprising particles of metal oxide having a median volume particle diameter in the range from 24 to 42 nm, dispersed in a medium which comprises a mixture of (i) at least one polar material having an interfacial tension of less than 30 mNm⁻¹, and (ii) at least one siloxane fluid, to produce a sunscreen having improved skin feel.

The invention still further provides the use of a dispersion comprising particles of metal oxide having a median volume particle diameter in the range from 24 to 42 nm, dispersed in a medium which comprises a mixture of (i) at least one polar material having an interfacial tension of less than 30 mNm⁻¹, and (ii) at least one siloxane fluid, in the manufacture of a transparent sunscreen having improved skin feel.

Preferably the metal oxide used in the present invention comprises an oxide of titanium, zinc or iron, and most preferably the metal oxide is titanium dioxide.

The preferred titanium dioxide particles comprise anatase and/or rutile crystal form. The titanium dioxide in the particles preferably comprises a major portion of rutile, more preferably greater than 60% by weight, particularly greater than 70%, and especially greater than 80% by weight of rutile. The titanium dioxide in the particles preferably comprises in the range from 0.01% to 5%, more preferably 0.1% to 2%, and particularly 0.2% to 0.5% by weight of anatase. In addition, the titanium dioxide in the particles preferably comprises less than 40%, more preferably less than 30%, and particularly less than 25% by weight of amorphous titanium dioxide. The basic particles may be prepared by standard procedures, such as using the chloride process, or by the sulphate process, or by hydrolysis of an appropriate titanium compound such as titanium oxydichloride or an organic or inorganic titanate, or by oxidation of an oxidisable titanium compound, e.g. in the vapour state. The titanium dioxide particles are preferably prepared by the hydrolysis of a titanium compound, particularly of titanium oxydichloride.

The particles of metal oxide used in the present invention are preferably hydrophobic. The hydrophobicity of the metal oxide can be determined by pressing a disc of metal oxide powder, and measuring the contact angle of a drop of water placed thereon, by standard techniques known in the art. The contact angle of a hydrophobic metal oxide is preferably greater than 50°.

The metal oxide particles are preferably coated in order to render them hydrophobic. Suitable coating materials are water-repellent, preferably organic, and include fatty acids, preferably fatty acids containing 10 to 20 carbon atoms, such as lauric acid, stearic acid and isostearic acid, salts of the above fatty acids such as sodium salts and aluminium salts, fatty alcohols, such as stearyl alcohol, and silicones such as polydimethylsiloxane and substituted polydimethylsiloxanes, and reactive silicones such as methylhydrosiloxane and polymers and copolymers thereof. Stearic acid and/or salt thereof is particularly preferred. The organic coating may be applied using any conventional process. Typically, metal oxide particles are dispersed in water and heated to a temperature in the range 50° C. to 80° C. A fatty acid, for example, is then deposited on the metal oxide particles by adding a salt of the fatty acid (e.g. sodium stearate) to the dispersion, followed by an acid. Alternatively, the metal oxide particles can be mixed with a solution of the water-repellent material in an organic solvent, followed by evaporation of the solvent. In an alternative embodiment of the invention, the water-repellant material can be added directly to the composition according to the present invention, during preparation thereof, such that the hydrophobic coating is formed in situ. Generally, the particles are treated with up to 25%, more preferably in the range from 3% to 20%, particularly 6% to 17%, and especially 10% to 15% by weight of organic material, preferably fatty acid, calculated with respect to the metal oxide core particles.

Alternatively or additionally, the particles of metal oxide may have an inorganic coating. For example, metal oxide particles, such as titanium dioxide, may be coated with oxides of other elements such as oxides of aluminium, zirconium or silicon, or mixtures thereof such as alumina and silica as disclosed in GB-2205088-A, the teaching of which is incorporated herein by reference. The preferred amount of inorganic coating is in the range from 2% to 25%, more preferably 4% to 20%, particularly 6% to 15%, and especially 8% to 12% by weight, calculated with respect to the weight of metal oxide core particles. The inorganic coating may be applied using techniques known in the art. A typical process comprises forming an aqueous dispersion of metal oxide particles in the presence of a soluble salt of the inorganic element whose oxide will form the coating. This dispersion is usually acidic or basic, depending upon the nature of the salt chosen, and precipitation of the inorganic oxide is achieved by adjusting the pH of the dispersion by the addition of acid or alkali, as appropriate.

In a preferred embodiment of the invention, the metal oxide particles are coated with both an inorganic and an organic coating, either sequentially or as a mixture. It is preferred that the inorganic coating, preferably alumina, is applied first followed by the organic coating, preferably fatty acid and/or salt thereof. Thus, preferred metal oxide particles for use in the present invention comprise (i) In the range from 60% to 98%, more preferably 65% to 95%, particularly 70% to 80%, and especially 72% to 78% by weight of metal oxide, preferably titanium dioxide, with respect to the total weight of the particles, (ii) In the range from 0.5% to 15%, more preferably 2% to 12%, particularly 5% to 10%, and especially 6% to 9% by weight of inorganic coating, preferably alumina, with respect to the total weight of the particles, and (iii) in the range from 1% to 21%, more preferably 4% to 18%, particularly 7% to 15%, and especially 9% to 12% by weight of organic coating, preferably fatty acid and/or salt thereof, with respect to the total weight of the particles. Such metal oxide particles provide a surprising combination of both improved photostability and dispersibility.

The individual or primary metal oxide particles are preferably acicular in shape and have a long axis (maximum dimension or length) and short axis (minimum dimension or width). The third axis of the particles (or depth) is preferably approximately the same dimensions as the width.

The mean length by number of the primary metal oxide particles is suitably in the range from 50 to 90 nm, preferably 55 to 77 nm, more preferably 55 to 73 nm, particularly 60 to 70 nm, and especially 60 to 65 nm. The mean width by number of the particles is suitably in the range from 5 to 20 nm, preferably 8 to 19 nm, more preferably 10 to 18 nm, particularly 12 to 17 nm, and especially 14 to 16 nm. The primary titanium dioxide particles preferably have a mean aspect ratio d₁:d₂ (where d₁ and d₂, respectively, are the length and width of the particle) in the range from 2.0 to 8.0:1, more preferably 3.0 to 6.5:1, particularly 4.0 to 6.0:1, and especially 4.5 to 5.5:1. The size of the primary particles can be suitably measured using electron microscopy. The size of a particle can be determined by measuring the length and width of a filler particle selected from a photographic image obtained by using a transmission electron microscope.

The metal oxide particles suitably have a mean crystal size (measured by X-ray diffraction as herein described) in the range from 4 to 10 nm, preferably 5 to 9 nm, more preferably 5.5 to 8.5 nm, particularly 6 to 8 nm, and especially 6.5 to 7.5 nm.

The size distribution of the crystal size of the metal oxide particles can be important, and suitably at least 30%, preferably at least 40%, more preferably at least 50%, particularly at least 60%, and especially at least 70% by weight of the metal oxide particles have a crystal size within one or more of the above preferred ranges for the mean crystal size.

When formed into a dispersion according to the present invention, the particulate metal oxide suitably has a median volume particle diameter (equivalent spherical diameter corresponding to 50% of the volume of all the particles, read on the cumulative distribution curve relating volume % to the diameter of the particles—often referred to as the “D(v,0.5)” value)) (hereinafter referred to as dispersion particle size), measured as herein described, in the range from 24 to 42 nm, preferably 27 to 39 nm, more preferably 29 to 37 nm, particularly 31 to 35 nm, and especially 32 to 34 nm.

The size distribution of the metal oxide particles in dispersion can also be an Important parameter in obtaining, for example, a sunscreen product having the required properties. In a preferred embodiment suitably less than 10% by volume of metal oxide particles have a volume diameter of more than 13 nm, preferably more than 11 nm, more preferably more than 10 nm, particularly more than 9 nm, and especially more than 8 nm below the median volume particle diameter. In addition, suitably less than 16% by volume of metal oxide particles have a volume diameter of more than 11 nm, preferably more than 9 nm, more preferably more than 8 nm, particularly more than 7 nm, and especially more than 6 nm below the median volume particle diameter. Further, suitably less than 30% by volume of metal oxide particles have a volume diameter of more than 7 nm, preferably more than 6 nm, more preferably more than 5 nm, particularly more than 4 nm, and especially more than 3 nm below the median volume particle diameter.

Also, suitably more than 90% by volume of metal oxide particles have a volume diameter of less than 30 nm, preferably less than 27 nm, more preferably less than 25 nm, particularly less than 23 nm, and especially less than 21 nm above the median volume particle diameter. In addition, suitably more than 84% by volume of metal oxide particles have a volume diameter of less than 19 nm, preferably less than 18 nm, more preferably less than 17 nm, particularly less than 16 nm, and especially less than 15 nm above the median volume particle diameter. Further, suitably more than 70% by volume of metal oxide particles have a volume diameter of less than 8 nm, preferably less than 7 nm, more preferably less than 6 nm, particularly less than 5 nm, and especially less than 4 nm above the median volume particle diameter.

Dispersion particle size of the metal oxide particles described herein may be measured by electron microscope, coulter counter, sedimentation analysis and static or dynamic light scattering. Techniques based on sedimentation analysis are preferred. The median particle size may be determined by plotting a cumulative distribution curve representing the percentage of particle volume below chosen particle sizes and measuring the 50th percentile. The median particle volume diameter and particle size distribution of the metal oxide particles in dispersion is suitably measured using a Brookhaven particle sizer, as herein described.

In a particularly preferred embodiment of the invention, the metal oxide particles have a BET specific surface area, measured as described herein, of greater than 40, more preferably in the range from 50 to 100, particularly 60 to 90, and especially 65 to 75 m²/g.

The metal oxide particles used in the present invention exhibit improved transparency suitably having an extinction coefficient at 524 nm (E₅₂₄), measured as herein described, in the range from 0.4 to 1.2, preferably 0.5 to 1.1, more preferably 0.6 to 1.0, particularly 0.7 to 0.9, and especially 0.75 to 0.85 l/g/cm. In addition, the metal oxide particles suitably have an extinction coefficient at 450 nm (E₄₅₀), measured as herein described, in the range from 0.8 to 2.2, preferably 1.0 to 2.0, more preferably 1.2 to 1.8, particularly 1.3 to 1.7, and especially 1.4 to 1.6 l/g/cm.

The metal oxide particles exhibit effective UV absorption, suitably having an extinction coefficient at 360 nm (E₃₆₀), measured as herein described, in the range from 5 to 11, preferably 6 to 10, more preferably 6.5 to 9.5, particularly 7 to 9, and especially 7.5 to 8.5 l/g/cm. The metal oxide particles also suitably have an extinction coefficient at 308 nm (E₃₀₈), measured as herein described, in the range from 40 to 52, preferably 42 to 50, more preferably 43 to 49, particularly 44 to 48, and especially 45 to 46 l/g/cm.

The metal oxide particles suitably have a maximum extinction coefficient E(max), measured as herein described, in the range from 57 to 70, preferably 58 to 68, more preferably 59 to 66, particularly 60 to 64, and especially 61 to 62 l/g/cm. The metal oxide particles suitably have a % (max), measured as herein described, in the range from 270 to 286, preferably 272 to 284, more preferably 274 to 282, particularly 276 to 280, and especially 277 to 278 nm.

The metal oxide particles suitably exhibit reduced whiteness, preferably having a change in whiteness ΔL of a sunscreen product containing the particles, measured as herein described, of less than 3, more preferably in the range from 0.5 to 2.5, and particularly 1.0 to 2.0. In addition, a sunscreen product containing the particles preferably has a whiteness index, measured as herein described, of less than 100%, more preferably in the range from 10% to 80%, particularly 20% to 60%, and especially 30% to 50%.

The metal oxide particles suitably have reduced photogreying, preferably having a photogreying index, measured as herein described, of less than 15, more preferably in the range from 1 to 10, particularly 2 to 7, and especially 3 to 5.

In a dispersion according to the present invention, the metal oxide particles are stable to aggregation. The metal oxide particles in the dispersion are relatively uniformly dispersed and resistant to settling out on standing, but if some settling out does occur, the particles can be easily redispersed by simple agitation.

The dispersing medium used in the present invention comprises a mixture of at least one organic polar material having an interfacial tension compared to water of less than 30 mNm⁻¹ and at least one siloxane fluid. The polar material suitably has an interfacial tension compared to water in the range from 2 to 28, preferably 3 to 25, more preferably 10 to 22, particularly 15 to 20, and especially 17 to 19 mNm⁻¹.

The ratio of polar material to siloxane fluid present in the dispersion is suitably in the range from 10 to 90:10 to 90, preferably 20 to 80:20 to 80, more preferably 30 to 70:30 to 70, particularly 40 to 60:40 to 60, and especially 45 to 55:45 to 55% by weight.

Suitable polar materials comprise, or are selected from the group consisting of, C12-15 alkyl benzoate, caprylic/capric triglyceride, cetearyl isononanoate, ethylhexyl isostearate, ethylhexyl palmitate, isononyl isononanoate, isopropyl isostearate, isopropyl myristate, isostearyl isostearate, isostearyl neopentanoate, octyldodecanol, pentaerythrityl tetraisostearate, PPG-15 stearyl ether, triethylhexyl triglyceride, dicaprylyl carbonate, ethylhexyl stearate, helianthus annus (sunflower) seed oil, isopropyl palmitate, octyldodecyl neopentanoate, propylene glycol isostearate, cetyl ethylhexanoate, cetearyl octanoate, hexyl laurate, isodecyl neopentanoate, isopropyl palmitate, decyl oleate, and isostearyl alcohol. Mixtures of any two or more of the above polar materials may be used. Preferred polar materials include isononyl isononanoate, triethylhexyl triglyceride, cetyl ethylhexanoate, hexyl laurate, isopropyl isostearate and/or propylene glycol isostearate. Isopropyl isostearate and/or propylene glycol isostearate are particularly preferred.

Any suitable siloxane fluid or silicone oil can be used, a principal requirement being cosmetic acceptability. Suitable siloxane fluids include those shown in the following table;

| INCI Name | Other Technical Names |
|---|---|
| Cyclomethicone | Methylcyclopolysiloxane |
| Cyclotrisiloxane | Hexamethylcyclotrisiloxane |
| Cyclotetrasiloxane | Octamethylcyclotetrasiloxane |
| Cyclopentasiloxane | Decamethylcyclopentasiloxane |
| Cyclohexasiloxane | Dodecamethylcyclohexasiloxane |
| Cycloheptasiloxane | Tetradecamethylcycloheptasiloxane |
| Dimethicone | Dimethylpolysiloxane, Dimethyl silicone, |
| | Highly polymerized methyl polysiloxane, |
| | Methyl polysiloxane |
| Diphenyl dimethicone | |
| Lauryl dimethicone | |
| Phenyl dimethicone | |
| Trimethylsiloxysilicate | |
| Diisostearyl | |
| trimethylolpropane | |
| siloxy silicate | |
| Dilauroyl | |
| trimethylolpropane | |
| siloxy silicate | |
| Phenyl trimethicone | Phenyltris(trimethylslloxy)silane |
| Triphenyl trimethicone | |
| Disiloxane | Hexamethyldisiloxane, Bis(trimethylsilyl) |
| | ether |
| Dimethiconol | Dihydroxypolydimethylsiloxane |

Preferred siloxane fluids include dimethylpolysiloxane, dimethyl silicone, highly polymerized methyl polysiloxane, and methyl polysiloxane, (known generically as dimethicone); cyclic oligomeric dialkylsiloxanes, such as the cyclic oligomers of dimethylsiloxane (known generically as cyclomethicone); dimethylsiloxane linear oligomers or polymers having a suitable fluidity; phenyltris(trimethylsiloxy)silane (also known as phenyltrimethicone); and mixtures thereof. Particularly preferred siloxane fluids are cyclomethicone and/or dimethicone.

The siloxane fluid preferably has a kinematic viscosity of less than 100, more preferably less than 50, particularly less than 30, and especially less than 10 cSt.

The dispersion according to the present invention may also contain a dispersing agent in order to improve the properties thereof. The dispersing agent is suitably present in the range from 1% to 25%, preferably 2 to 20%, more preferably 3% to 15%, particularly 4 to 10%, and especially 6 to 8% by weight based on the total weight of metal oxide particles. A surprising feature is that dispersions can be produced at relatively low concentrations of dispersing agent.

A further surprising feature of metal oxide dispersions according to the present invention, is that a dispersing medium comprising a siloxane fluid can be used without using a polysiloxane dispersing agent.

Suitable dispersing agents include substituted carboxylic acids, soap bases and polyhydroxy acids. Typically the dispersing agent can be one having a formula X.CO.AR in which A is a divalent bridging group, R is a primary secondary or tertiary amino group or a salt thereof with an acid or a quaternary ammonium salt group and X is the residue of a polyester chain which together with the —CO— group is derived from a hydroxy carboxylic acid of the formula HO—R′—COOH. As examples of typical dispersing agents are those based on ricinoleic acid, hydroxystearic acid, hydrogenated castor oil fatty acid which contains in addition to 12-hydroxystearic acid small amounts of stearic acid and palmitic acid. Dispersing agents based on one or more polyesters or salts of a hydroxycarboxylic acid and a carboxylic acid free of hydroxy groups can also be used. Compounds of various molecular weights can be used. Other suitable dispersing agents are those monoesters of fatty acid alkanolamides and carboxylic acids and their salts. Alkanolamides are based on ethanolamine, propanolamine or aminoethyl ethanolamine for example. Alternative dispersing agents are those based on polymers or copolymers of acrylic or methacrylic acids, e.g. block copolymers of such monomers. Other dispersing agents of similar general form are those having epoxy groups in the constituent radicals such as those based on the ethoxylated phosphate esters. The dispersing agent can be one of those commercially referred to as a hyper dispersant. Polyhydroxystearic acid is a particularly preferred dispersing agent.

An advantage of the present invention is that dispersions can be produced which contain at least 35%, preferably at least 40%, more preferably at least 45%, particularly at least 50%, especially at least 55%, and generally up to 70% by weight of the total weight of the dispersion, of metal oxide particles.

A particular surprising feature of dispersions according to the present invention is that they can have low viscosity, even at high metal oxide concentrations, such as at least 40% by weight of the total weight of the dispersion. The dispersion suitably has (i) a low shear viscosity, measured as described herein, of less than 25,000, more preferably less than 15,000, more preferably in the range from 500 to 10,000, particularly 1,000 to 5,000, and especially 2,000 to 3,000 mPa·s, and/or (ii) a high shear viscosity, measured as described herein, of less than 6,000, more preferably less than 4,000, more preferably in the range from 100 to 3,000, particularly 500 to 2,000, and especially 800 to 1,500 mPa·s.

The dispersions according to the present invention exhibit improved skin feel particularly when used to produce sunscreen products. Conventional sunscreen products exhibit a ‘greasy’ afterfeel on the skin. Dispersions and sunscreen products according to the present invention surprisingly have improved skin feel, measured as herein described, for example conferring a significantly less greasy afterfeel and/or a reduced amount of residue.

A composition, preferably a sunscreen product, containing the dispersion of metal oxide particles defined herein suitably has a Sun Protection Factor (SPF), measured as herein described, of greater than 10, preferably greater than 15, more preferably greater than 20, particularly greater than 25, and especially greater than 30 and up to 50.

The dispersions of the present invention are useful as ingredients for preparing sunscreen compositions, especially in the form of emulsions. The compositions may further contain conventional additives suitable for use in the intended application, such as conventional cosmetic ingredients used in sunscreens. The particulate metal oxide as defined herein, may provide the only ultraviolet light attenuators in a sunscreen product according to the invention, but other sunscreening agents, such as other metal oxides and/or other organic materials may also be added. For example, the preferred titanium dioxide particles defined herein may be used in combination with other existing commercially available titanium dioxide and/or zinc oxide sunscreens. Suitable organic sunscreens for use with a composition according to the invention include p-methoxy cinnamic acid esters, salicylic acid esters, p-amino benzoic acid esters, non-sulphonated benzophenone derivatives, derivatives of dibenzoyl methane and esters of 2-cyanoacrylic acid. Specific examples of useful organic sunscreens include benzophenone-1, benzophenone-2, benzophenone-3, benzophenone-6, benzophenone-8, benzophenone-12, isopropyl dibenzoyl methane, butyl methoxy dibenzoyl methane, ethyl dihydroxypropyl PABA, glyceryl PABA, octyl dimethyl PABA, octyl methoxycinnamate, homosalate, octyl salicylate, octyl triazone, octocrylene, etocrylene, menthyl anthranilate, and 4-methylbenzylidene camphor.

In this specification the following test methods have been used:

### 1) Crystal Size Measurement of Metal Oxide Particles

Crystal size was measured by X-ray diffraction (XRD) line broadening. Diffraction patterns were measured with Cu Kα radiation in a Siemens D5000 diffractometer equipped with a Sol-X energy dispersive detector acting as a monochromator. Programmable slits were used to measure diffraction from a 12 mm length of specimen with a step size of 0.02° and step counting time of 3 sec. The data was analysed by fitting the diffraction pattern between 22 and 48° 2θ with a set of peaks corresponding to the reflection positions for rutile and, where anatase was present, an additional set of peaks corresponding to those reflections. The fitting process allowed for removal of the effects of instrument broadening on the diffraction line shapes. The value of the weight average mean crystal size was determined for the rutile 110 reflection (at approximately 27.4° 2θ) based on its integral breadth according to the principles of the method of Stokes and Wilson (B. E. Warren, “X-Ray Diffraction”, Addison-Wesley, Reading, Mass., 1969, pp 254-257).

### 2) Median Particle Volume Diameter and Particle Size Distribution of Metal Oxide Particles in Dispersion

A dispersion of metal oxide particles was produced by mixing 6.3 g of polyhydroxystearic acid with 51.85 g of polar material (e.g. isopropyl isostearate or propylene glycol isostearate) and 51.85 g of siloxane fluid (e.g. cyclomethicone), and then adding 90 g of metal oxide into the solution. The mixture was passed through a horizontal bead mill, operating at approximately 2100 r.p.m. and containing zirconia beads as grinding media for 15 minutes. The dispersion of metal oxide particles was diluted to between 30 and 40 g/l by mixing with isopropyl myristate. The diluted sample was analysed on the Brookhaven BI-XDC particle sizer in centrifugation mode, and the median particle volume diameter and particle size distribution measured.

### 3) BET Specific Surface Area of Metal Oxide Particles

The single point BET specific surface area was measured using a Micromeritics Flowsorb II 2300.

### 4) Change in Whiteness and Whiteness Index

A sunscreen formulation was coated on to the surface of a glossy black card and drawn down using a No 2 K bar to form a film of 12 μm wet thickness. The film was allowed to dry at room temperature for 10 minutes and the whiteness of the coating on the black surface (L_{F}) measured using a Minolta CR300 colourimeter. The change in whiteness ΔL was calculated by subtracting the whiteness of the substrate (L_{S}) from the whiteness of the coating (L_{F}). The whiteness index is the percentage change in whiteness ΔL compared to a standard titanium dioxide (=100% value) (Tayca MT100T (ex Tayca Corporation)).

### 5) Photogreying Index

A metal oxide dispersion (15% by weight of metal oxide particles in C12-15 alkyl benzoate) was placed inside a 6 cm×3 cm acrylic cell (containing a 2 cm×1.5 cm space), and the cell made air tight by clamping a glass slide over the top, ensuring that no air bubbles were present. The initial whiteness (L_{I}) was measured using a Minolta CR300 colourimeter. The cell was then placed on a turntable revolving at 30 rpm and exposed to UV light for 2 hours (a UV lamp containing 4 TL29D, 16/09 tubes mounted 12 cm from the cell), and the whiteness (L_{T}) remeasured. The photogreying Index ΔL=L_{I}−L_{T}.

### 6) Sun Protection Factor

The Sun Protection Factor (SPF) of a sunscreen formulation was determined using the in vitro method of Diffey and Robson, J. Soc. Cosmet. Chem. Vol. 40, pp 127-133, 1989.

### 7) Extinction Coefficients

0.1 g sample of a metal oxide dispersion was diluted with 100 ml of cyclohexane. This diluted sample was then further diluted with cyclohexane in the ratio sample:cyclohexane of 1:19. The total dilution was 1:20,000. The diluted sample was then placed in a spectrophotometer (Perkin-Elmer Lambda 2 UV/VIS Spectrophotometer) with a 1 cm path length and the absorbance, of UV and visible light measured. Extinction coefficients were calculated from the equation A=E.c.l, where A=absorbance, E=extinction coefficient in litres per gram per cm, c=concentration in grams per litre, and l=path length in cm.

### 8) Interfacial Tension

Interfacial Tension between the polar material or siloxane fluid dispersing medium and water was measured by the Du Nuoy Ring method at 25° C.

### 9) Viscosity

Viscosity of the metal oxide dispersion was measured using a Bohlin CVO rheometer using a C25 concentric cylinder geometry at 25° C. Samples were pre-sheared at a shear rate of 1000 s⁻¹ for 30 s prior to measurement. Measurements were carried out immediately upon completion of the pre-shear period, by applying an initial shear stress of 0.03 Pa which was ramped up to 1000 Pa over a period of 180 s. Viscosity at low (0.01 s⁻¹) and high (100 s⁻¹) shear rates were recorded.

The invention is illustrated by the following non-limiting examples.

### EXAMPLES

### Example 1

1 mole of titanium oxydichloride in acidic solution was reacted with 3 moles of NaOH in aqueous solution. After the initial reaction period, the temperature was increased to above 70° C., and stirring continued. The reaction mixture was neutralised by the addition of aqueous NaOH, and allowed to cool below 70° C. An alkaline solution of sodium aluminate was then added, equivalent to 10.5% by weight Al₂O₃ on TiO₂ weight. The temperature was maintained below 70° C. during the addition. The temperature was increased to above 70° C., and sodium stearate equivalent to 13.5% by weight sodium stearate on weight of TiO₂ was added. The dispersion was neutralised by adding hydrochloric acid solution. The resultant slurry was filtered, washed with demineralised water, dried in an oven, and then micropulverised to produce particulate titanium dioxide.

A dispersion was produced by mixing 6.3 g of polyhydroxystearic acid with 51.85 g of isopropyl isostearate and 51.85 g of cyclomethicone, and then adding 90 g of titanium dioxide produced above into the solution. The mixture was passed through a horizontal bead mill, operating at approximately 2100 r.p.m. and containing zirconia beads as grinding media for 15 minutes.

The dispersion was subjected to the test procedures described herein, and the titanium dioxide exhibited the following properties:

i) Mean crystal size=7 nm,

### ii) Extinction Coefficients;

| E₅₂₄ | E₄₅₀ | E₃₀₈ | E₃₆₀ | E(max) | λ(max) |
|---|---|---|---|---|---|
| 0.9 | 1.6 | 45.6 | 7.6 | 60.1 | 280 |

### Example 2

The procedure of Example 1 was repeated except that propylene glycol isostearate was used instead of isopropyl isostearate.

The dispersion was subjected to the test procedures described herein, and the titanium dioxide exhibited the following properties:

i) Mean crystal size=7 nm,
ii) Low shear viscosity=2,480 mPa·s, and high shear viscosity=1,000 mPa·s.
iii) Extinction Coefficients;

| E₅₂₄ | E₄₅₀ | E₃₀₈ | E₃₆₀ | E(max) | λ(max) |
|---|---|---|---|---|---|
| 0.7 | 1.5 | 46.7 | 6.9 | 62.2 | 279 |

### Example 3

The titanium dioxide dispersion produced in Example 1 was used to prepare a sunscreen product having the following composition.

% w/w

Phase A:

| | |
|---|---|
| Cyclomethicone (Pentamer) | 7.75 |
| ESTOL 1543 (trade mark, ex Uniqema) | 9.75 |
| DC 2502 Fluid (ex Dow Corning) | 2.0 |
| Titanium Dioxide dispersion produced in Example 1 | 12.0 |

Phase B:

| | |
|---|---|
| ARLATONE 2121 (trade mark, ex Uniqema) | 4.5 |
| Keltrol RD (ex Nutrasweet Kelco) | 0.2 |
| Veegum Ultra (ex R T Vanderbilt) | 0.8 |
| Rewoderm S1333 (ex Witco) | 0.2 |
| D-Panthenol USP (ex Roche) | 0.8 |
| Magnesium Sulfate | 0.7 |
| Propylene Glycol | 4.0 |
| Water | to 100 |

Phase C:

| | |
|---|---|
| Phenonip (ex Nipa) | 0.4 |

### Procedure:

1) The water and Arlatone 2121 were mixed, and heated to 80° C. with moderate stirring, until the Arlatone 2121 was fully dispersed.
2) The remaining phase B Ingredients were added with stirring, maintaining the temperature at 75-80° C.
3) Phase A Ingredients were heated to 75-80° C.
4) Phase A was added to phase to B with stirring. The mixture was homogenised for 2 minutes.
5) The mixture was cooled to 45° C. with stirring. Phase C was added.
6) Cooling, with stirring, was continued to room temperature.

### Example 4

The titanium dioxide dispersion produced in Example 2 was used to prepare a sunscreen product having the following composition.

% w/w

Phase A:

| | |
|---|---|
| Cyclomethicone (Pentamer) | 6.0 |
| KF6028 | 3.0 |
| Arlacel 183V (trade mark, ex Uniqema) | 0.6 |
| Octyl methoxycinnamate | 7.5 |
| Isoamyl p-methoxycinnamate | 2.0 |
| Octyl salicylate | 3.2 |

Phase B:

| | |
|---|---|
| PRICERINE 9091 (trade mark, ex Uniqema) | 2.0 |
| 1,3 butylene glycol | 3.0 |
| Magnesium sulfate | 0.8 |
| Water | to 100 |

Phase C:

| | |
|---|---|
| Bentonite gel VS5PC-V | 6.0 |

Phase D :

| | |
|---|---|
| Titanium Dioxide dispersion produced in Example 2 | 12.5 |

Phase E:

| | |
|---|---|
| Phenonip (ex Nipa) | 0.4 |

### Procedure:

1) Phase A and B ingredients were mixed together separately.
2) Phases A and B were heated separately, up to 70° C.
3) Phase B was added into phase A whilst homogenising for 5 minutes.
4) The mixture was cooled to 55-60° C.
5) Phases C, D and E were added to the mixture. The mixture was homogenised for 2 to 3 minutes.
6) Cooling was continued to room temperature.

The resulting emulsion was found to exhibit excellent skin feel properties having a non greasy after-feel with minimal skin residue. The emulsion had an SPF of 40.1.

### Example 5

The titanium dioxide dispersion produced in Example 2 was used to prepare a sunscreen product (sun balm) having the following composition.

| | % w/w |
|---|---|
| Beeswax | 8.5 |
| Hydrogenated olive oil stearyl esters | 6.0 |
| Vaseline | 8.0 |
| Shea butter | 3.0 |
| Behenyl alcohol | 2.5 |
| MONASIL PCA (trade mark, ex Uniqema) | 3.0 |
| Octyl methoxycinnamate | 7.5 |
| Isoamyl p-methoxycinnamate | 2.0 |
| Cyclomethicone | 4.0 |
| PRISORINE 3631 (trade mark, Uniqema) | 6.5 |
| PRISORINE 2039 (trade mark, Uniqema) | 7.0 |
| Titanium Dioxide dispersion produced in Example 2 | 25.0 |
| Silica | 5.0 |
| Pigment (green) | 2.0 |
| Tapioca pure | 10 |

### Procedure:

1) All of the ingredients were mixed together and heated up to 80° C.
2) The mixture was stirred until it was well dispersed.
3) The mixture was cooled to 60° C. and poured into a container.
4) Cooling was continued to room temperature.

The resulting sun balm was found to exhibit a non greasy application and dry powdery afterfeel. The sun balm had an SPF of 47.6.

The above examples illustrate the improved properties of a dispersion and sunscreen product according to the present invention.

1. A dispersion comprising particles of metal oxide having a median volume particle diameter in the range from 24 to 42 nm, dispersed in a medium which comprises a mixture of (i) at least one polar material having an interfacial tension of less than 30 mNm⁻¹, and (ii) at least one siloxane fluid.

2. A dispersion according to claim 1 wherein the metal oxide particles have a mean crystal size in the range from 4 to 10 nm.

3. A dispersion according to claim 1 wherein at least 40% by weight of metal oxide particles have a crystal size within the range 5 to 9 nm.

4. A dispersion according to claim 1 wherein less than 16% by volume of metal oxide particles have a volume diameter of more than 9 nm below the median volume particle diameter.

5. A dispersion according to claim 1 wherein less than 30% by volume of metal oxide particles have a volume diameter of more than 5 nm below the median volume particle diameter.

6. A dispersion according to claim 1 wherein more than 84% by volume of metal oxide particles have a volume diameter of less than 17 nm above the median volume particle diameter.

7. A dispersion according to claim 1 wherein more than 70% by volume of metal oxide particles have a volume diameter of less than 6 nm above the median volume particle diameter.

8. A dispersion according to claim 1 wherein the metal oxide particles have an extinction coefficient at 524 nm in the range from 0.4 to 1.2 l/g/cm.

9. A dispersion according to claim 1 wherein the metal oxide particles have an extinction coefficient at 360 nm in the range from 5 to 11 l/g/cm.

10. A dispersion according to claim 1 wherein the metal oxide particles have an extinction coefficient at 308 nm in the range from 40 to 52 l/g/cm.

11. A dispersion according to claim 1 wherein the metal oxide particles have (i) a median volume particle diameter in the range from 29 to 37 nm, and/or (ii) less than 10% by volume of particles having a volume diameter of more than 11 nm below the median volume particle diameter, and/or (iii) less than 16% by volume of particles having a volume diameter of more than 8 nm below the median volume particle diameter, and/or (iv) less than 30% by volume of particles having a volume diameter of more than 5 nm below the median volume particle diameter, and/or (v) more than 90% by volume of particles having a volume diameter of less than 27 nm above the median volume particle diameter, and/or (vi) more than 84% by volume of particles having a volume diameter of less than 17 nm above the median volume particle diameter, and/or (vii) more than 70% by volume of particles having a volume diameter of less than 6 nm above the median volume particle diameter.

12. A dispersion according to claim 1 wherein the particles of metal oxide have an extinction coefficient at 524 nm in the range from 0.5 to 1.1 l/g/cm, an extinction coefficient at 450 nm in the range from 1.0 to 2.0 l/g/cm, an extinction coefficient at 360 nm in the range from 6 to 10 l/g/cm, an extinction coefficient at 308 nm in the range from 44 to 48 l/g/cm, a maximum extinction coefficient in the range from 60 to 64 l/g/cm, and a λ(max) in the range from 274 to 282 nm.

13. A dispersion according to claim 1 wherein the polar material has an interfacial tension in the range from 3 to 25 mNm⁻¹.

14. A dispersion according to claim 1 wherein the ratio of polar material to siloxane fluid is in the range from 30 to 70:30 to 70% by weight.

15. A dispersion according to claim 1 wherein the polar material is selected from the group consisting of isononyl isononanoate, triethylhexyl triglyceride, cetyl ethylhexanoate, hexyl laurate, isopropyl isostearate and propylene glycol isostearate.

16. A dispersion according to claim 1 wherein the siloxane fluid is selected from the group consisting of cyclomethicone and dimethicone.

17. A dispersion according to claim 1 comprising a dispersing agent comprising a non-polysiloxane.

18. A dispersion according to claim 17 wherein the dispersing agent consists of at least one non-polysiloxane.

19. A dispersion according to claim 17 wherein the dispersing agent comprises polyhydroxystearic acid.

20. A dispersion according to claim 1 comprising at least 45% by weight of metal oxide particles.

21. A dispersion according to claim 1 having (i) a low shear viscosity in the range from 500 to 10,000 mPa·s, and/or (ii) a high shear viscosity in the range from 100 to 3,000 mPa·s.

22. A dispersion comprising particles of metal oxide having a median volume particle diameter in the range from 24 to 42 nm, dispersed in a medium which comprises a mixture of (i) at least one polar material selected from the group consisting of, C12-15 alkyl benzoate, caprylic/capric triglyceride, cetearyl isononanoate, ethylhexyl isostearate, ethylhexyl palmitate, isononyl isononanoate, isopropyl isostearate, isopropyl myristate, isostearyl isostearate, isostearyl neopentanoate, octyldodecanol, pentaerythrityl tetraisostearate, PPG-15 stearyl ether, triethylhexyl triglyceride, dicaprylyl carbonate, ethylhexyl stearate, helianthus annus (sunflower) seed oil, isopropyl palmitate, octyldodecyl neopentanoate, propylene glycol isostearate, cetyl ethylhexanoate, cetearyl octanoate, hexyl laurate, isodecyl neopentanoate, isopropyl palmitate, decyl oleate, and isostearyl alcohol; and (ii) at least one siloxane fluid selected from the group consisting of, dimethylpolysiloxane, dimethyl silicone, highly polymerized methyl polysiloxane, and methyl polysiloxane, cyclic oligomeric dialkylsiloxanes, dimethylsiloxane linear oligomers or polymers, and phenyltris(trimethylsiloxy)silane.

23. A sunscreen product formed from a dispersion comprising particles of metal oxide having a median volume particle diameter in the range from 24 to 42 nm, dispersed in a medium which comprises a mixture of (i) at least one polar material having an interfacial tension of less than 30 mNm⁻¹, and (ii) at least one siloxane fluid.

24. A sunscreen product comprising particles of metal oxide having a median volume particle diameter in the range from 24 to 42 nm, and (i) at least one polar material selected from the group consisting of isopropyl isostearate and propylene glycol isostearate, and (ii) at least one siloxane fluid selected from the group consisting of cyclomethicone and dimethicone.

25. The use of a dispersion comprising particles of metal oxide having a median volume particle diameter in the range from 24 to 42 nm, dispersed in a medium which comprises a mixture of (i) at least one polar material having an interfacial tension of less than 30 mNm⁻¹, and (ii) at least one siloxane fluid, to produce a sunscreen having improved skin feel.

26. The use of a dispersion comprising particles of metal oxide having a median volume particle diameter in the range from 24 to 42 nm, dispersed in a medium which comprises a mixture of (i) at least one polar material having an interfacial tension of less than 30 mNm⁻¹, and (ii) at least one siloxane fluid, in the manufacture of a transparent sunscreen having improved skin feel.
